(19) United States
(12) Patent Application Publication
(10) Pub. No.: US 2023/0268063 A1
(43) Pub. Date: Aug. 24, 2023

(54) Time Out Fail-Safe Device

(71) Applicants: Robert Rodela, Corpus Christi, TX (US); Luis Reyes, McAllen, TX (US)

(72) Inventors: Robert Rodela, Corpus Christi, TX (US); Luis Reyes, McAllen, TX (US)

(21) Appl. No.: 18/112,451

(22) Filed: Feb. 21, 2023

Related U.S. Application Data
(60) Provisional application No. 63/312,275, filed on Feb. 21, 2022.

(51) Int. Cl. G16H 40/20; G16H 40/40; G16H 40/63

(52) U.S. Cl. CPC G16H 40/20; G16H 40/40; G16H 40/63

(57) ABSTRACT

A device may include a light source, an interface, and a microcontroller unit (MCU). The light source may emit light toward a treatment surface to produce an optical indicator. The interface may be configured to receive input data to change a status of a time out procedure. The MCU may be coupled to the light source and the interface. The MCU may be configured to receive first input data from the interface and control the light source to produce a first optical indicator including one or more first parameters in response to the first input data; receive second input data and control the light source to produce a second optical indicator including one or more second parameters in response to the second input data; and receive third input data and turn off the light source in response to the third input data.

### CROSS-REFERENCE TO RELATED APPLICATION(S)

This application is a non-provisional of and claims priority to U.S. Provisional Patent Application No. 63/312,275 filed on Feb. 21, 2022 and entitled “Time Out Fail-Safe Device”, which is incorporated herein by reference in its entirety.

### FIELD

The present disclosure is generally related to accurate identification and verification of a surface (surgical site, auto part, or other surface) to be processed, and more particularly to a time out fail-safe device configured to project an optical indicator onto a surface to be worked on as part of a verification process.

### BACKGROUND

Prior to performing a procedure, especially a medical procedure, it may be important to verify the procedure to be performed and the person or object on which the procedure is to be performed. In some environments, such as an automotive environment, performing the wrong procedure on the wrong vehicle or replacing the wrong part may be costly in terms of monetary costs and in terms of lost time. In the medical environment, performing the wrong procedure or performing the right procedure on the wrong site or the wrong patient may be incredibly costly, both monetarily and with respect to the health of the patient.

Few medical errors are as terrifying as those that involve doctors performing a medical procedure on the wrong body part, performing the wrong procedure, or performing a scheduled procedure on the wrong patient. To prevent wrong site, wrong procedure, wrong person medical events, in 2004 the Joint Commission on Accreditation of Healthcare Organizations adopted a Universal Protocol, which requires performing a “time out” prior to beginning the medical procedure. The time out is a planned pause before beginning the procedure to allow time for the medical team to review important aspects of the procedure with all involved personnel in order to prevent mistakes. Despite the widespread implementation of the Universal Protocol since 2004, multiple reports have documented continued occurrence of wrong site and wrong patient procedures.

If wrong site or wrong procedure events can still occur in medical settings that require high attention to detail, wrong site and wrong procedure events may also occur in other settings, and particularly in training settings.

### SUMMARY

Embodiments of a time out device are described below that may include a light-emitting interface configured to project one or more shapes or words onto a selected site to facilitate an optical verification prior to proceeding. In a medical environment, the time out device may include a housing formed from an antimicrobial and antibacterial material and may be oriented and adjusted to project one or more shapes or letters onto a treatment site on a patient. In an example, the time out device may be coupled to or integrated with an overhead light system and may be configured to project “Time Out” in red letters on the treatment site. Once the time out procedure is completed, the users may interact with the time out device to change the status, causing the time out device to project “Time Out” in green letters or to project a different word or a shape on the treatment site to indicate that the time out procedure has been completed successfully. Once the treatment begins (e.g., radiation is applied or the surgeon begins to make an incision), the user may interact with the time out device to change the state, which may cause the time out device to turn off the emitted light.

In a training environment, such as an environment for training a technician, an electrician, a pilot, a mechanic, or another type of individual, the time out device may be configured by a user to project one or more letters, words, or shapes onto a surface. The surface may include a portion of a control panel, an automotive part, a circuit, or another object. During a verification process, the time out device may illuminate one or more portions of a control panel, parts, or objects. Once the verification process is complete, the user may interact with the time out device to change the state, causing the time out device to change one or more of the letters, the words, the shapes, the colors, or any combination thereof. Other implementations are also possible.

In some implementations, a device may include a light source, an interface, and a microcontroller unit (MCU). The light source may emit light toward a surface to produce an optical indicator. The interface may be configured to receive input data to change a status of a time out procedure. The MCU may be coupled to the light source and the interface. The MCU may be configured to receive first input data from the interface and control the light source to produce a first optical indicator including one or more first parameters in response to the first input data; receive second input data and control the light source to produce a second optical indicator including one or more second parameters in response to the second input data; and receive third input data and turn off the light source in response to the third input data. The input data may be received from an interface based on interactions with a user or determined based on signals from another system, such as a patient monitoring system, a patient records system, or another system, which may be tracking activities by the various participants.

In other implementations, a device may be configured to facilitate a time out verification procedure. The device may include one or more light sources configured to emit light toward a surface, producing an optically visible indicator on the surface. The device may include a microcontroller configured to control the one or more light sources to produce a first indicator at a start of the time out verification procedure and a second optical indicator at successful completion of the time out verification procedure. During the time out verification procedure, one or more users may verify one or more details of the procedure and may interact with an interface of the device to indicate successful completion of the time out verification procedure.

In still other implementations, a device may be configured to facilitate a time out verification procedure. The device may include one or more light sources configured to emit light toward a surface to produce an optically visible indicator. The device may include an interface coupled to the one or more light sources to receive input data from user interactions directly with the interface or from interactions with another system in communication with the interface. The device may include a microcontroller unit configured to control the one or more light sources to produce a first optical indicator at a start of the time out verification procedure and a second optical indicator at successful completion of the time out verification procedure in response to receiving the input data.

### BRIEF DESCRIPTION OF THE DRAWINGS

The detailed description is set forth with reference to the accompanying figures. In the figures, the left most digit(s) of a reference number identifies the figure in which the reference number first appears. The use of the same reference numbers in different figures indicates similar or identical items or features.

FIG. 1 depicts a flow diagram of a method of performing a medical time out using a time out device, in accordance with certain embodiments of the present disclosure.

FIG. 2 depicts a block diagram of a system including a time out device, in accordance with certain embodiments of the present disclosure.

FIG. 3 depicts a diagram of a system including a time out device including an adjustable arm and an attachment mechanism configured to couple to a light assembly, in accordance with certain embodiments of the present disclosure.

FIG. 4 depicts a diagram of a system including a time out device including an adjustable arm and an attachment mechanism configured to couple to a pivot arm of a light assembly, in accordance with certain embodiments of the present disclosure.

FIG. 5A depicts a diagram of a system including a time out device, an adjustable arm, and an attachment mechanism implemented as a clamp, in accordance with certain embodiments of the present disclosure.

FIG. 5B depicts a diagram of a system including a time out device, an adjustable arm, and a base to provide stability, in accordance with certain embodiments of the present disclosure.

FIGS. 6A and 6B depicts diagrams of a surgical site being prepared for surgery while the time out device projects the words “TIME OUT” onto the surgical site, in accordance with certain embodiments of the present disclosure.

FIG. 7 depicts a flow diagram of a method of performing a time out check list using the time out device, in accordance with certain embodiments of the present disclosure.

FIG. 8 depicts a block diagram of a system configured to automate a time out procedure by prompting a team for information, verifying the information, and automatically controlling a time out device, in accordance with certain embodiments of the present disclosure.

FIG. 9 depicts a flow diagram of a method of automating a time out procedure, in accordance with certain embodiments of the present disclosure.

While implementations are described in this disclosure by way of example, those skilled in the art will recognize that the implementations are not limited to the examples or figures described. The figures and detailed description thereto are not intended to limit implementations to the form disclosed, but on the contrary, the intention is to cover all modifications, equivalents, and alternatives falling within the spirit and scope as defined by the appended claims. The headings used in this disclosure are for organizational purposes only and are not meant to limit the scope of the description or the claims. As used throughout this application, the word “may” is used in a permissive sense (in other words, the term “may” is intended to mean “having the potential to”) instead of in a mandatory sense (as in “must”). Similarly, the terms “include”, “including”, and “includes” mean “including, but not limited to”.

### DETAILED DESCRIPTION OF ILLUSTRATIVE EMBODIMENTS

Embodiments of systems, methods, and devices are described below that may enforce or otherwise facilitate a time out procedure within which one or more participants may review various parameters to verify details associated with the procedure to ensure that the correct procedure is to be performed on the correct part. The time out procedure may be employed in a variety of contexts including surgical environments, maintenance environments, repair environments, training environments, or other environments in which confirmation may be relevant or important.

In some implementations, the time out device may use one or more optically visible indicators to enforce or facilitate a time out procedure. In an example, the time out device may be configured to project an optical indicator onto a selected surface to facilitate a time out procedure. The selected surface may depend on the context of the time out procedure, and the optical indicator may depict a stop signal or other signal that may cause the participants to pause before proceeding to allow time to verify that the correct procedure is about to be performed on the correct part or surface. In general, the time out procedure is a planned or desired pause before beginning the procedure to review important aspects of the procedure with all involved personnel to prevent mistakes. The time out procedure may require all involved personnel to participate in the review to verify the correct procedure, on the correct person or thing, and at the correct site. By enforcing a time out procedure, the device may assist the participants in making sure that they do not waste time or materials and that they do not perform an unwanted or unnecessary procedure on the wrong part, system, or patient.

In a medical context, the selected surface may be a treatment site on a patient, such as surgical site, a site to be exposed to radiation, and so on. In an automotive context, the selected surface may include a car part, such as a side panel, a belt, a hose, a compressor, an engine part, or another automotive component. In computer training context, the site may include one or more ports on a rear portion of a router device, a server, or other computing device. In an training context, the site may include cables, circuits, devices, and so on. Other examples are also possible.

The time out device may be configured to project a symbol, a shape, a message, or any combination thereof onto a site that one or more individuals (such as a medical team) are expected to work on. In some implementations, the time out device may project the phrase “TIME OUT” directly onto the surface, reminding the users to stop and review the procedures, the site, and other information to make sure that the procedure, the setup, the tools, the site location, and other parameters to make sure that everything is correct before the user or the team begins. In the medical context, this time out may prevent surgical participants from performing the wrong procedure, operating on the wrong patient, performing the correct procedure on the right patient but in the wrong area, and so on.

In the following discussion, the time out device is primarily described with respect to a surgical time out and with respect use in a surgical environment; however, the time out device may be used in training situations or on objects other than patients. Such objects can include construction walls to be painted or demolished, automotive parts, control panels, electrical cabling, computing devices, other devices, or any combination thereof.

It is important to understand that the surgical time out is typically performed immediately prior to starting a procedure, as a final recapitulation immediately prior to surgery. The time out was one of several verification procedures that were introduced by the Joint Commission on Hospital Accreditation as part of the Universal Protocol for Preventing Wrong Site, Wrong Procedure, Wrong Person Surgery in 2003. The verification procedures included conducting a pre-procedure verification process, marking the procedure site, and performing a time out immediately before starting the invasive procedure to resolve any questions and concerns and to verify all surgical information prior to beginning. Even with adoption of these procedures, wrong site, wrong procedure, and wrong person surgical mistakes still occur.

Embodiments of the devices and methods described below may reduce or eliminate such mistakes by maintaining an optical marker on the surgical site during the pre-surgical time out verification process. The visual nature of the optical marker and the process of deactivating the optical marker may require active steps by one or more users, enforcing a level of engagement with the time out verification process, which may serve to prevent mistakes. An example of a method of using a time out device to enforce a surgical time out.

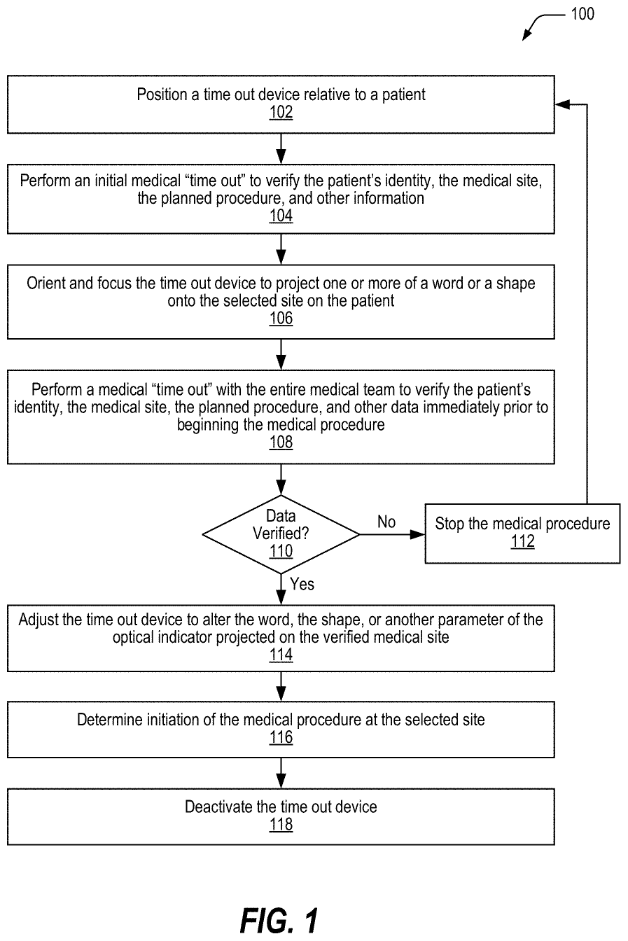

FIG. 1 depicts a flow diagram of a method 100 of performing a medical or surgical time out using a time out device, in accordance with certain embodiments of the present disclosure. The medical or surgical time out may be a formal verification procedure that may precede performance of a medical procedure, such as a radiation treatment, a surgical procedure, and so on. The surgical time out may be performed by all the participants in the process to confirm and verify the site, the procedure, and the patient.

At 102, the method 100 may include positioning a time out device relative to a patient. In an example, a nurse or technician may position the time out device near the patent. In some implementations, the time out device may be integrated with an overhead surgical lighting system. In other implementations, the time out device may include an attachment mechanism that may be coupled one of the overhead lights, to the ceiling, to an adjustment arm of one of the lights, or to a movable base that sits on the floor. The time out device may include or may be coupled to one or more of an adjustable arm, a flexible arm, a pivot, a hinge, or another component that may enable adjustment of the position of the time out device relative to the patient. Further, the time out device may include a control interface that may include one or more buttons, switches, sliders, knobs, or other user-selectable control elements that may be accessed by the nurse or technician to adjust the displayed letters, shapes, words, colors, and so on.

In some implementations, all the components of the time out device may be formed from or coated by an antimicrobial and antibacterial material that is authorized for use in surgical environments. In some implementations, the control interface may include one or more communication interfaces configured to receive radio frequency signals from a remote-control device, a smartphone, a tablet computer, another computing device, or any combination thereof, which signals may include control instructions that may adjust the adjust the displayed letters, shapes, words, colors, and so on. In some instances, the control elements or control signals may be used to adjust the focus to display an optical indicator on a selected site on the patient.

At 104, the method 100 may include performing an initial medical “time out” to verify the patient's identity, the medical site, the planned procedure, and other information. In an example, the technician, the anesthesiologist, and one or more nurses may perform the initial medical time out while doing surgical preparations and in some instances before the surgeon has entered the operating room.

At 106, the method 100 may include orientating and focusing the time out device to project one or more of a word or a shape onto the selected site on the patient. In an example, the nurse or technician may adjust one or more of the length an adjustable arm, a curvature of the adjustable arm, the orientation of a light-emitting lens, a focus of the light-emitting lens, other parameters, or any combination thereof in order to project an optical indicator onto the selected site on the patient. The selected site may include the surgical site or the site to receive treatment.

At 108, the method 100 may include performing a medical “time out” with the entire medical team to verify and confirm the patient's identity, the medical site, the planned procedure, and other data immediately prior to beginning the medical procedure. The medical time out may include confirming the name, gender, and birthdate of the patient, identifying the medical team in the operating room and specifying their respective roles, confirming the procedure to be performed, confirming the site on the patient for performing the procedure, and so on. The medical time out may include a well-defined checklist of information to be verified before proceeding, and all the members of the medical team may participate in the time out verification process.

At 110, the method 100 determines if the data is verified. If the patient's name or birthdate differs from the scheduled patient, for example, the data is not verified. Similarly, if the procedure, surgical site, or other information differs from the data in the patient's chart or in the associated systems, the method 100 may include stopping the medical procedure, at 112. This stoppage constitutes a safety measure to prevent an error.

Depending on the basis for the error, such as the wrong surgical site or other errors, the method 100 may include repositioning the time out device relative to the patient, at 102. The method 100 may then proceed as previously described by performing an initial medical “time out” to verify or confirm the patient's identity, the medical site, the medical procedure, and other information, at 104.

Returning to 110, if the data is verified, the method 100 may include adjusting the time out device to alter the word, the shape, or another parameter of the optical indicator projected on the verified medical site, at 114. In some instances, the color of the shape or word projected onto the selected site may change, for example, from red (indicating stop) to green (indicating go). In some implementations, upon successful verification, the technician or nurse may interact with one or more user-selectable control options on an interface of the time out device to change the status of the time out. In an example, a first button press may activate the time out device to project the phrase “TIME OUT” in red letters onto the selected site. A second button press may cause the time out device to change the color of the phrase from red to green while continuing to project the letters onto the selected site. A third button press may turn off the time out device. Other control variations are also possible. Additionally, in some implementations, the wording may change as the time out device changes state. Other implementations are also possible.

At 116, the method 100 may include determining initiation of the medical procedure at the selected site. In some implementations, initiation of the medical procedure may include activating a radiation element to direct radiation at the selected site. In other implementations, initiation of the medical procedure may include the surgeon making an incision at the selected site.

At 118, the method 100 may include deactivating the time out device. In some implementations, the time out device may be deactivated manually, such as by a nurse or technician pressing a button on the time out device, a button on a remote control, a button on an interface of a computing device, interacting with a computing system, or via another means.

It should be understood that the method 100 at 118 and 116 may be reversed so that the deactivation of the time out device in 118 occurs before determining initiation in 116. Other adjustments may also be made without departing from the scope of the disclosure.

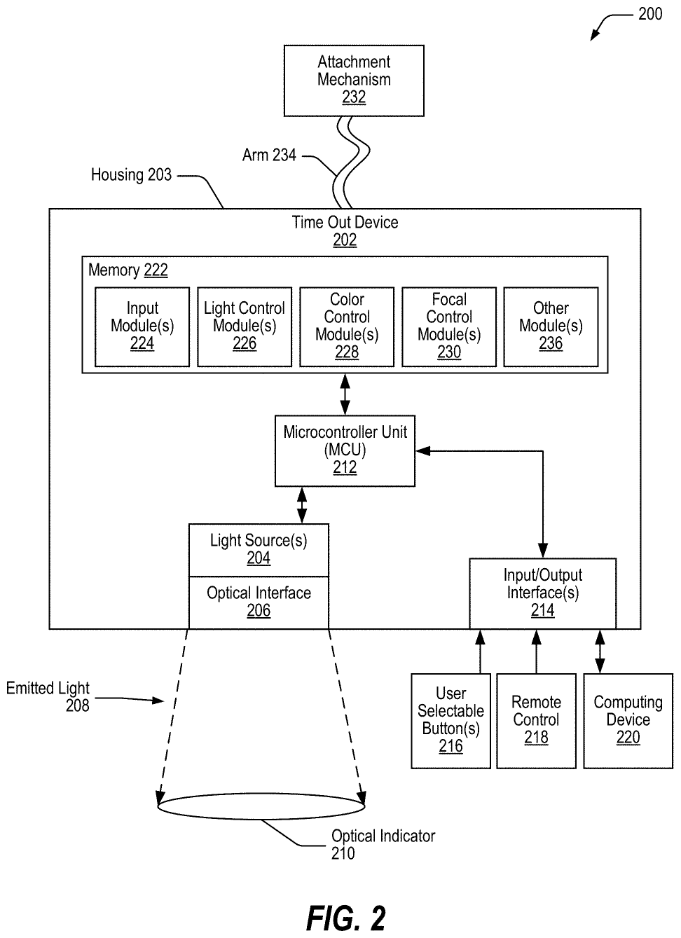

While the method 100 in FIG. 1 is directed to a medical procedure, it should be understood that the time out device may be employed in a variety of contexts to provide a non-invasive optical indicator that may be used as a double-check to ensure that the correct procedure is being applied to the correct site. In some implementations, the time out device may be programmable to allow a user to configure the device to present a predetermined word or phrase, to vary the colors, and so on. One possible implementation of the time out device is described below with respect to FIG. 2.

FIG. 2 depicts a block diagram of a system 200 including a time out device 202, in accordance with certain embodiments of the present disclosure. The time out device 202 may include a housing 203 configured to enclose and secure the various components and to isolate the components from the environment. In some implementations, the housing 203 may be formed from antimicrobial or antibacterial material, which may be approved for use in sterile environments.

The time out device 202 may include one or more light sources 204 (such as one or more light-emitting diodes, a halogen lamp, another light source, or any combination thereof) configured to emit light. The time out device 202 may include an optical interface 206 that may be configured to direct and optionally focus the emitted light 208 onto a surface to provide an optical indicator 210. In some implementations, the optical interface 206 may include a translucent lens or cover to allow the light to pass through and to protect the light sources 204 from the ambient environment. In some implementations, the optical interface 208 may include one or more focal lenses that may be controlled to focus the emitted light. In some implementations, the optical interface 208 may include a mask or other opaque feature configured to selectively block light to allow formation of a pattern as the optical indicator 210. Other implementations are also possible.

The time out device 202 may include a microcontroller unit (MCU) 212, which may include a processor configured to execute processor-readable instructions to control one or more of the light sources 204 or the optical interface 206.

The time out device 202 may include one or more input/output (I/O) interfaces 214, which may be configured to receive input selections or input data from one or more user-selectable buttons 216, a remote control 218, or a computing device 220 (such as a smartphone or tablet computer). In some implementations, to trigger the time out device to display an optical indicator as part of a time out procedure, a user may press a button, and the user may press the same or another button to disable the time out device when the time out procedure is successfully completed. The button may be one of the one or more user-selectable buttons 216, a button of the remote control 218, or a user-selectable control option presented on an output interface (such as a touchscreen or other display) of the computing device 220. The user may press the button a first time to cause the time out device 202 to activate the light source 204 to project a first optical indicator 210 having a first phrase, a first color, a first shape, or any combination thereof onto a surface, such as a selected surgical site. After the time out procedure has been completed to confirm the details of the procedure, the user may press the button a second time to cause the time out device 202 to control the light source 204 to project a second phrase, a second color, a second shape, or any combination thereof onto the surface. Once the procedure is started, the user may press the button a third time to turn off the light source 204.

In some implementations, the time out device 202 may include a microcontroller unit (MCU) 212, which may be coupled to the I/O interfaces 214 and to the light source 204. The MCU 212 may be configured to control operation of the light source 204 based on input data received from the I/O interface 214, based on processor-readable instructions, or both. The MCU 212 may include a processing circuit that may be configured to execute processor-readable instructions, which may be stored in a memory 222 of the time out device 202.

The memory 222 may include one or more input modules 224 that may cause the MCU 212 to receive data from the I/O interface 214. The data may include input data corresponding to a user-selectable button 216 or input data from the remote control 218 or from a computing device 220. In some implementations, the data may include input data, processor-readable instructions, or any combination thereof from the computing device 220.

The memory 222 may include one or more light control modules 226 that may cause the MCU 212 to control the one or more light sources 204 to provide an optical indicator 210, which may be light formed in a shape, a letter, a word, or a phrase and which may include one or more colors. The light control modules 226 may cause the MCU 212 to control or otherwise alter the shape of the light to provide a selected optical indicator 210. In some implementations, the optical indicator 210 may include the phrase “TIME OUT”, a stop sign shape, another shape, or any combination thereof.

The memory 222 may include one or more color control modules 228 that may cause the MCU 212 to control the frequency of the light emitted by the light sources 204 to selectively alter one or more colors of the optical indicator 210. In some implementations, the MCU 212 may control the light sources 204 to emit an optical indicator 210 that is red. The MCU 212 may control the light sources 204 to emit the optical indicator 210 in a selected color in response to input data received from the I/O interface 214.

The memory 222 may include one or more focal control modules 230 that may cause the MCU 230 to adjust one or more of the light sources 204 or the optical interface 206 to control one or more of the size or focus of the optical indicator 210. In an example, the positioning of the time out device 202 (distance, angle, and so on) may vary relative to the surface, and the focal control modules 230 may cause the MCU 212 to adjust the size and focus to project the optical indicator 210 onto the selected surface.

The memory 222 may also include other modules 236, which may provide other functionality or processes. In an example, the other modules 236 may include an update module that may be configured to receive processor-readable instructions (new instructions or upgraded instructions) from the computing device 220 and that may install the received processor-readable instructions in the memory 222 for subsequent execution. The other modules 236 may include instructions that may be executed by the MCU 212 to perform other functions. Other implementations are also possible.

In some implementations, the system 200 may include an attachment mechanism 232 that may be configured to couple to a structure, such as a light fixture, a handle, a ceiling mount structure, another structure, or any combination thereof. In some implementations, the attachment mechanism 232 may include a clamp, a clip, or another releasable attachment mechanism. In some implementations, the attachment mechanism 232 may include a base or other component configured to provide stability for the time out device 202. The system 200 may include an adjustable arm 234 that may include a first end coupled to the attachment mechanism 232 and a second end coupled to the time out device 202. The adjustable arm 234 may be flexible to adjust the position or orientation of the time out device 202. The adjustable arm 234 may telescope to extend or retract to adjust the distance between the time out device 202 and the surface onto which the optical indicator 210 is projected.

In a medical context, the attachment mechanism 232 may be fixed to the ceiling over the surgical table or onto a wall or other structure by screws or other fasteners. In another implementation, the attachment mechanism 232 may be releasable coupled to a light fixture, to a ceiling mount, to an adjustable arm of the light fixture, or to another structure. In other implementations, the attachment mechanism 232 may be replaced with a base or other structure configured to provide stability for the arm 234 and the time out device 202. Other implementations are also possible.

In some implementations, the time out device 202 may be integrated within the overhead surgical light. In other implementations, the time out device 202 may be coupled to the surgical light. In other types of environments, the time out device 202 may be coupled to any number of components or devices.

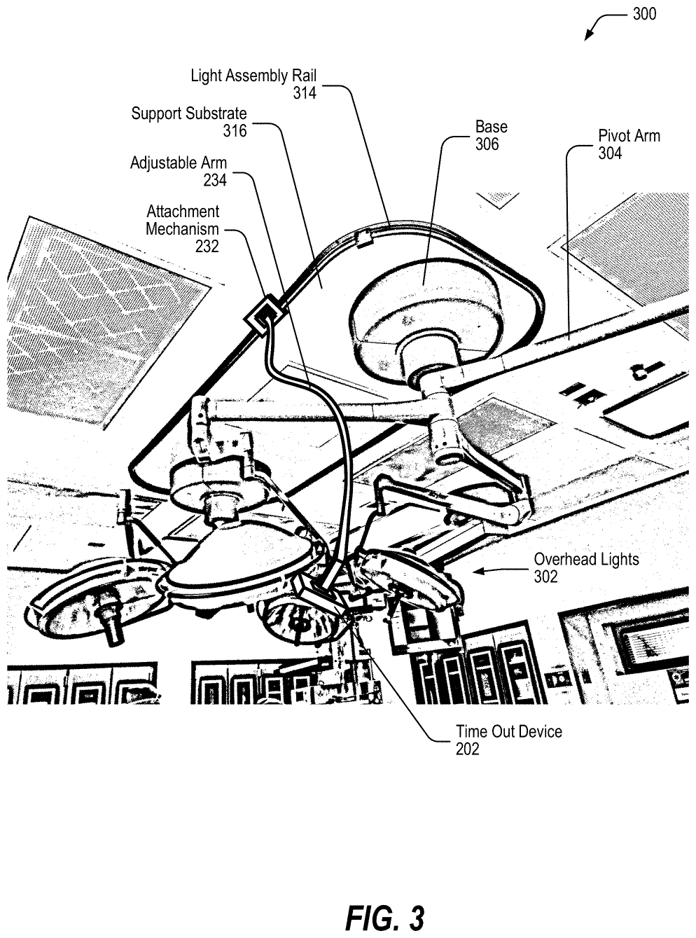

FIG. 3 depicts a diagram of a system 300 including a time out device 202 including an adjustable arm 234 and an attachment mechanism 232 configured to couple to a light assembly, in accordance with certain embodiments of the present disclosure. In the illustrated example of FIG. 3, the time out device 202 may be used in a medical context, such as a surgical unit, a radiological unit, a dentist office, and so on to project an optical indicator 210 onto a surface. The surface may include a surgical site, a radiological site, a tooth, or another surface on a patient.

In this example, the light assembly may include a one or more overhead lights 302, each of which may be coupled to a base 306 by a pivot arm 304. The pivot arm 304 may include one or more pivot points that allow for rotation of one portion of the arm relative to another portion to position the light 302. A user may adjust the position of each of the lights 302 and may orient each of the lights 302 to provide a selected illumination. The light assembly may include multiple bases 306, each of which may include multiple lights 302 and associated pivot arms 304. In this example, the light assembly may include a light assembly rail 314 or other structure that may be mounted to a support substrate 316, which may couple the bases 306 to the ceiling. Other implementations are also possible.

In the illustrated example, the attachment mechanism 232 may be configured to attach to an existing structure, such as the light assembly rail 314, the pivot arm 304, the light fixture 302, or another structure. The flexible arm 234 may be adjusted to position and at least partially orient the time out device 202.

It should be appreciated that the attachment mechanism 232 may be implemented in a variety of form factors that may enable releasable or fixed coupling to various structures and devices. The attachment mechanism 232 may provide support for securing the time out device 202 in a variety of environments. In an alternative implementation, the attachment mechanism may be attached to a leg, arm, surface, or other rigid structure of the operating table or chair. While FIG. 3 depicts the attachment mechanism 232 coupled to the light assembly rail 314 or the support substrate 316 near the ceiling, in the following example, the attachment mechanism 232 is shown as being coupled to one of the pivot arms 304.

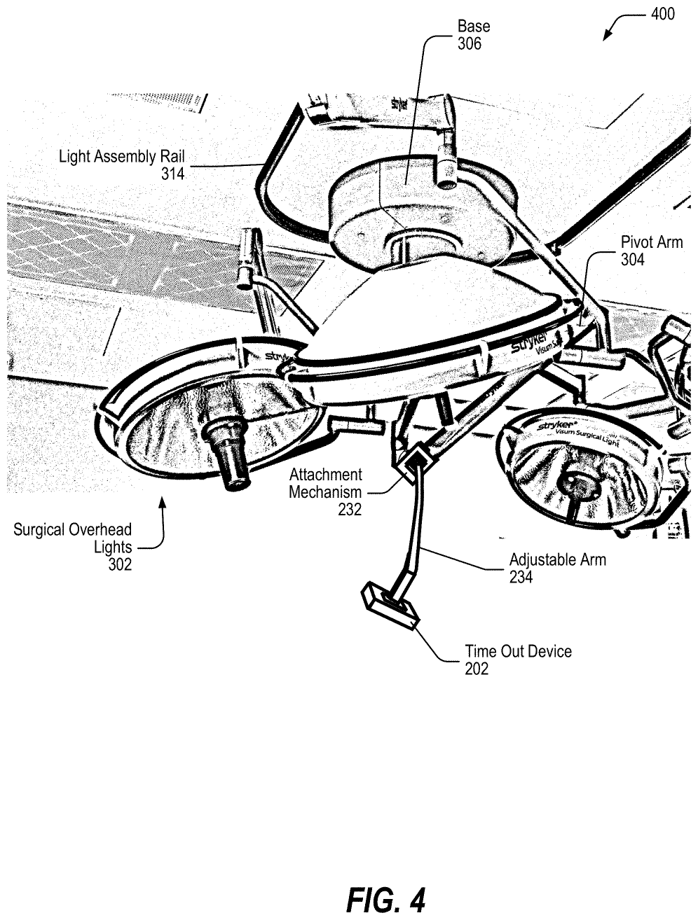

FIG. 4 depicts a diagram of a system 400 including a time out device 202 including an adjustable arm 234 and an attachment mechanism 232 configured to couple to a pivot arm 304 of a light assembly, in accordance with certain embodiments of the present disclosure. In this example, the adjustable arm 234 is coupled to the attachment mechanism 232, which is mounted to one of the pivot arms 304. The adjustable arm 234, in this example, may be smaller than or shortened relative to example depicted in FIG. 3. In some implementations, the adjustable arm 234 may be a telescoping arm that may be extended or retracted as needed. In other implementations, the adjustable arm 234 may be decoupled from the attachment mechanism 232 and from the time out device 202 and may be replaced with an adjustable arm 234 of a different length. Other implementations are also possible.

While the examples shown in FIGS. 3 and 4 illustrate implementations of the time out device 202 configured for use in a medical environment, it should be appreciated that the time out device 202 may be employed in other environments to project an optical indicator onto a surface.

In the examples of FIGS. 3 and 4, the time out device 202 is depicted as being coupled to the overhead light fixture. However, it should be appreciated that the time out device 202 may be incorporated within a larger fixture, such as the overhead light fixture.

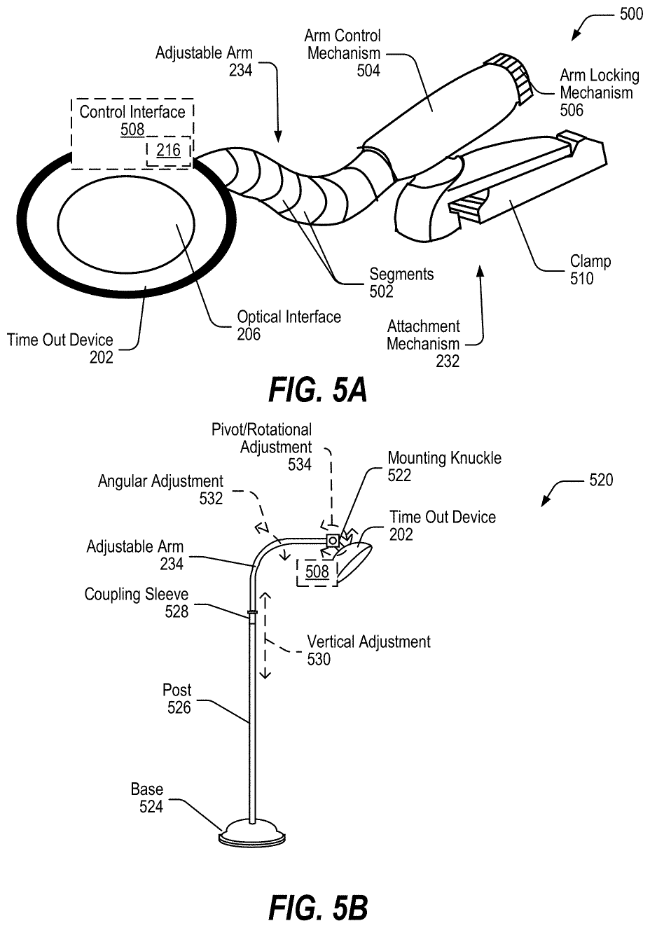

FIG. 5A depicts a diagram of a system 500 including a time out device 202, an adjustable arm 234, and an attachment mechanism 232 implemented as a clamp 502, in accordance with certain embodiments of the present disclosure. In this example, the adjustable arm 234 may be coupled between the time out device 202 and the attachment mechanism 232. The adjustable arm 234 may be comprised of a plurality of segments 502, that may shift or turn relative to one another to allow the adjustable arm 234 to bend. In some implementations, the segments 502 may be adjusted to a position and orientation and then may hold the position and without further manipulation. In other implementations, the adjustable arm 234 may include an arm control mechanism 504 that may include an arm locking mechanism 506 that may be turned, pushed, pulled, or otherwise manipulated to lock the adjustable arm 234 in a selected configuration. The attachment mechanism 232 may include a clamp 520 or other element coupled to the arm control mechanism 504.

In some implementations, the time out device 202 may include a control interface 508 (shown in phantom), which may include one or more user-selectable buttons or controls. The user may interact with the one or more buttons or controls of the control interface 508 to control the time out device 202 to project the optical indicator 210, optionally to alter the focus, the color, the shape, the letters, or any combination thereof, or to turn off the optical indicator 210.

In some implementations, the attachment mechanism 232 may be configured to engage a rail, a table, a handle, or other element to secure the time out device 202 and the adjustable arm 234. The user may then interact with the arm locking mechanism 506 and one or more of the adjustable arm 234 or the time out device 202 to adjust the position and orientation of the time out device 202 relative to a surface.

FIG. 5B depicts a diagram of a system 520 including a time out device 202, an adjustable arm 234, and a base 524 to provide stability, in accordance with certain embodiments of the present disclosure. In this example, the system 520 may be implemented in a configuration that is similar to a floor lamp. The base 524 may include a mass selected to provide stability for the system 520. A post 526 may be coupled to the base 524 and may extend away from the base 524 vertically. The post 526 may include a first end that is threaded and sized to fit and couple to a threaded opening in the base 524. A second end of the post 526 may include or may be coupled to a coupling sleeve 528 that may be configured to couple to the adjustable arm 234. In some implementations, post 526 may include two or more segments that may fit one within the other and that may be extended to provide a telescoping feature to allow for vertical adjustment 520 of the length of the post 526. Alternatively, a portion of the adjustable arm 234 may fit within the post 524 and may be drawn out to provide the vertical adjustment 230.

Additionally, the adjustable arm 234 may be bent or otherwise adjusted to provide an angular adjustment 532. A proximal end of the adjustable arm 234 may be coupled to or may fit within the post 526 and may be secured by the coupling sleeve 528. A distal end of the adjustable arm 234 may be coupled to the time out device 202 by a mounting knuckle 522. The mounting knuckle 522 may be configured to pivot, rotate, or otherwise turn relative to the distal end of the adjustable arm 234 to allow for directional adjustment of the time out device 202 as indicated by the pivot/rotational adjustment 534.

It should be appreciated that the vertical adjustment 530, the angular adjustment 532, and the pivot/rotation adjustment 534 may be incorporated in any of the implementations of the time out device 202 and the associated components and methods described above with respect to FIGS. 1-5B. The adjustable arm 234 in this implementation or in any of the other implementations may be flexible to enable a user to configure the shape and may be configured to maintain its shape. In this implementation and in other implementations described FIGS. 1-5B, the adjustable arm 234 may be configured to extend or retract to provide adjustability for the length. Additionally, the mounting knuckle 522 may be included between the adjustable arm 234 and the time out device 202 in any of the implementations described above with respect to FIGS. 1-5B. Other implementations are also possible.

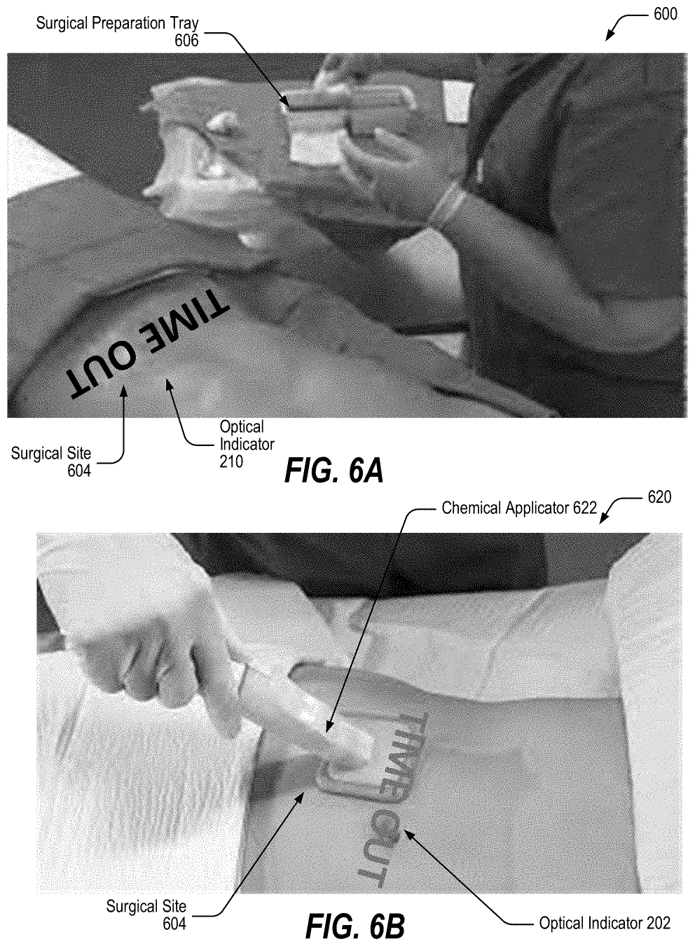

FIGS. 6A and 6B depicts diagrams of a surgical site being prepared for surgery while the time out device projects the words “TIME OUT” onto the surgical site, in accordance with certain embodiments of the present disclosure. In FIG. 6A, the time out device 202 is configured to apply the optical indicator 210 onto a surgical site 604. In this example, the optical indicator 210 includes the phrase “TIME OUT”, which may be projected in a selected color, such as red. The color or the wording may indicate that the medical team needs to perform a time out procedure prior to taking any action with respect to the patient. In the background, the nurse is holding a surgical preparation tray 606, which may include iodine and other chemicals to treat the surgical site 604.

In FIG. 6B, the nurse or another member of the medical team is using a chemical applicator 622 to apply iodine to the surgical site 604. The chemical applicator 622 may be used after the patient's identity, the procedure, and the surgical site have been confirmed as part of the time out procedure or checklist. Once the time out has been completed, a user may select one or more control options to alter a state of the time out device 202, which may cause the time out device to alter the color, the shape, the wording, or other parameters of the optical indicator 210.

In this example, the time out device 202 continues to project the phrase “TIME OUT” onto the surgical site 604, but the color is changed from red to green (or to a different color). In other embodiments, the wording may change, the shape may change, or other parameters of the optical indicator 210 may be changed to reflect the successful completion of the time out procedure. For example, the wording may be changed from “TIME OUT” to “TIME OUT Completed Successfully” or to some other phrase or indicator.

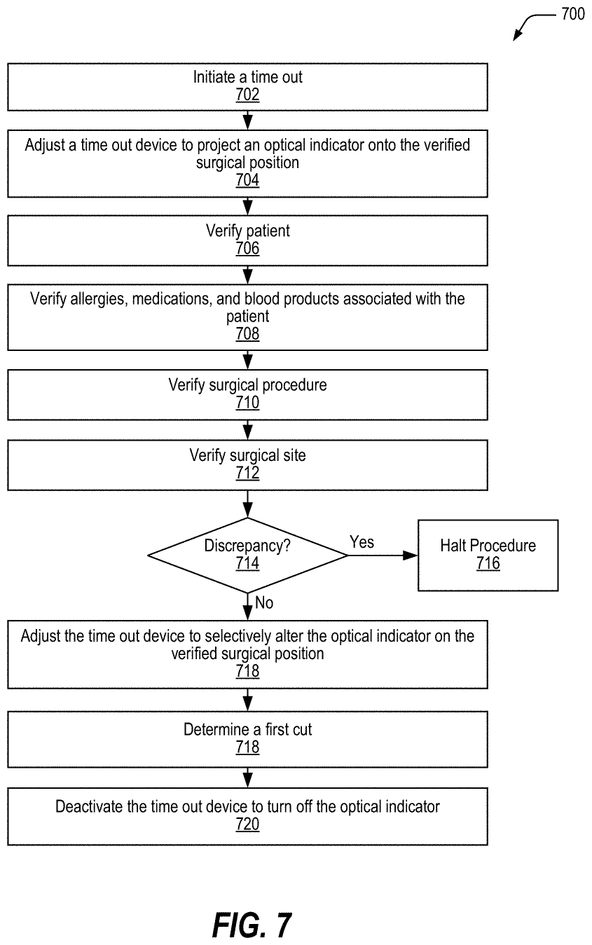

FIG. 7 depicts a flow diagram of a method 700 of performing a time out check list using the time out device, in accordance with certain embodiments of the present disclosure. The illustrated example is directed to implementation of a surgical or medical time out but could be readily adapted for use in other contexts or environments.

At 702, the method 700 may include initiating a time out. In some implementations, a time out may be initiated by one or more of a nurse, a doctor, an anesthesiologist, a medical technician, or other personnel, and one of them may orient and activate a time out device 202 to project an optical indicator 210 onto the patient. The optical indicator 210 may be indicative of the time out status of a procedure. The participants may then proceed with completion of a time out checklist. In some implementations, the team members who are to be involved in the procedure may determine the site or location on the patient at which the procedure is to be performed.

At 704, the method 700 may include adjusting a time out device 202 to project an optical indicator 210 onto the surgical site on the patient. In some implementations, a nurse or medical technician may activate the time out device 202 and may alter one or more of the position or orientation of the time out device 202 relative to the patient to project the optical indicator 210 onto the surgical site or location on the patient.

At 706, the method 700 may include verifying the patient. In some implementations, the patient may be verified by determining the patient's name, the patient's birthdate, appearance, and other identifying information. The information may be determined by comparing data in the medical chart of the patient, on a band attached to the patient, or from another source to one another and optionally to data stored in a patient database. In some instances, the information may be confirmed with the patient directly, such as prior to administering anesthesia.

At 708, the method 700 may include verifying allergies, medications, and blood products associated with the patient. The data may be verified by reviewing information on the patient's chart, by communicating directly with the patient, or by reviewing data in his or her medical record, which may be accessible via a computing device.

At 710, the method 700 may include verifying the surgical procedure. The personnel may confirm their respective understandings of the procedure to be performed. The personnel may confirm the surgical procedure with the patient, by reviewing data in the patient's medical records, by retrieving information in his or her medical chart, or any combination thereof. The confirmed information should match in the medical records and in the medical chart and should be consistent with the patient's understanding.

At 712, the method 700 may include verifying the surgical site (or medical site). The participants may each confirm their understanding of the intended site, which may be confirmed with one or more of the patient's chart, the patient's medical records in the computing system, or the patient.

At any of the steps 706 to 712, if the expected information does not match the patient's computerized medical records, the patient's chart, or the patient's understanding, the team members may halt the procedure and reconfirm the details. The team members may investigate any discrepancies to avoid an error with respect to one or more of a wrong procedure, a wrong patient, or a wrong site.

At 714, if there is a discrepancy between the medical records, the medical chart, the patient's understanding, or the understanding of the personnel with respect to the patient, the procedure, or the site of the surgery, the method 700 proceeds to 716 and the procedure is halted.

Otherwise, if there is no discrepancy at 714, the method 700 may include adjusting the time out device 202 to selectively alter the optical indicator 210 on the verified surgical position, at 718. In some instances, verification of the surgical site may result in a nurse or a technician adjusting the time out device 202 to position the optical indicator 210 on the verified surgical site. Further, adjusting the time out device 202 may include adjusting a color, a shape, one or more letters or words of the optical indicator 210 to provide an indication that a successful time out has been completed.

At 716, the method 700 may include determining a first cut (or a first step of a medical procedure). The first cut may be determined based on one or more of a user input to a control interface 508 of the time out device 202, input data from a remote control 218, input data from a computing device 220, a verbal input, an optical input, or any combination thereof. In a medical procedure other than surgery (such as a radiation treatment for cancer treatment), activation of the irradiating element may provide determination of the start of the procedure.

At 722, the method 700 may include deactivating the time out device 202 to turn off the optical indicator 210. In an example, once the procedure begins, the time out device 202 may be turned off by a nurse by pressing a button on the time out device 202, by pressing a button on remote control device 218, or by interacting with a computing device 220. In some instances, a computing system associated with the medical procedure may cause the time out device 202 to turn off. Other implementations are also possible.

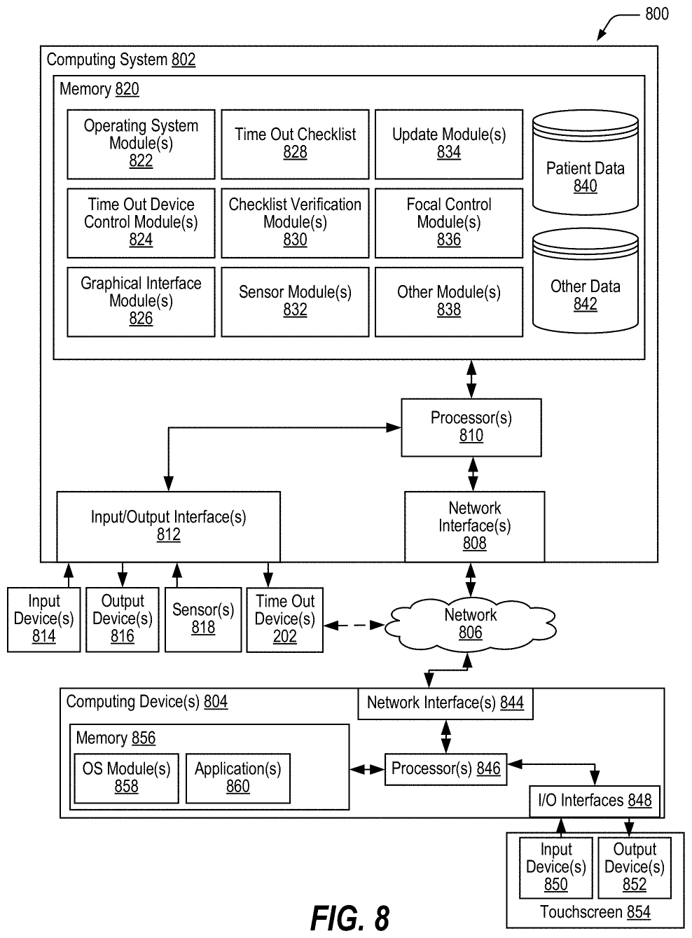

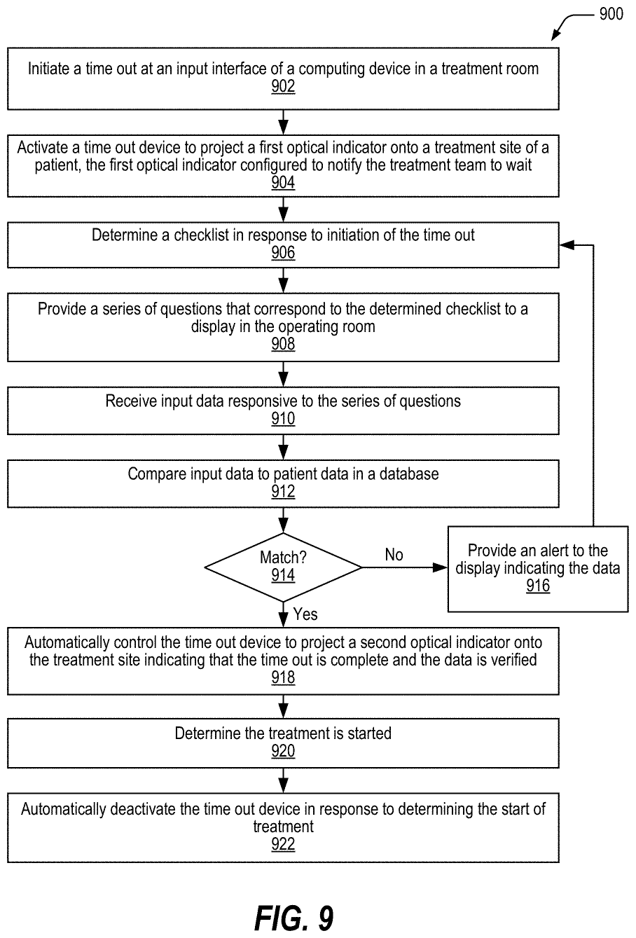

The examples provided above describe manual interactions with the time out device 202 to activate and direct the optical indicator 210 onto the site and to adjust one or more parameters of the optical indicator 210 when a time out procedure is successfully completed. In other implementations, such as those described below with respect to FIGS. 8 and 9, the time out device 202 may be coupled to or integrated with another computing system through wired connections or wireless communications links, and the computing system may provide a workflow to guide team through a standardized time out procedure while controlling operation of the time out device 202. An example of such a computing system is described below with respect to FIG. 8.

FIG. 8 depicts a block diagram of a system 800 configured to automate at least a portion of a time out procedure by prompting a team for information, verifying the information, and automatically controlling a time out device 202, in accordance with certain embodiments of the present disclosure. The system 800 may include a computing system 802, which may include a computer server, a personal computer, or another computing system. The computing system 802 may be integrated with or may operate various applications and systems, including calendaring applications, room schedules, human resources, patient processing, medical records, billing, and so on.

The computing system 802 may be configured to communicate with multiple time out devices 202, which may be distributed at various locations in which a time out procedure may be executed. In some implementations, the computing system 802 may automatically control the time out device 202 or may control the time out device in conjunction with user interactions with the computing device 804. The computing system 802 is described with respect to medical procedures but may be used in other contexts as previously described.

The computing system 802 that may be coupled to one or more other computing devices 804 via a network 806. In an example, the computing device 802 may be a server system within a medical office, a hospital, or other business. The computing device 802 may be an implementation of the computing device 220 in FIG. 2. The network 806 may be a private network of a medical facility (such as a doctor's office or a hospital, for example). The computing devices 804 may include smartphones, tablet computers, desktop computers, other computing devices, or any combination thereof.

The computing device 802 may include one or more network interfaces 808, which may communicate data to and receive data from the network 806. The computing device 802 may include one or more processors 808, which may be coupled to the network interfaces 808. The processors 810 may be configured to execute processor-readable instructions and to perform various operations.

The computing device 802 may include one or more input/output (I/O) interfaces 812 that may be configured to couple to one or more input devices 814, one or more output devices 816, one or more sensors 818, and optionally one or more time out devices 202. The input devices 814 may include one or more of a keyboard, a stylus, a mouse, a roller ball, a touch-sensitive interface, a camera, a microphone, a scanner, other input devices, or any combination thereof. The output devices 814 may include one or more of a display, a printer, a speaker, another output device, or any combination thereof. The one or more sensors 818 may include optical sensors, thermal sensors, moisture sensors, chemical sensors, other sensors, or any combination thereof.

The computing device 802 may include a memory 820, which may store processor-readable instructions and data. The memory 820 may include operating system modules 822 that may be executed by the processor 810 to control operation of various components, such as the interfaces 808 and 812.

The memory 820 may include one or more time out device control modules 824 that may cause the processor 810 to activate a time out device 202 through the I/O interface 812 or through the network 806. The time out device control modules 824 may cause the processor 810 to selectively control the color and the letters, shapes, words, or other aspects of an optical indicator 210 produced by the time out device 202. The device control modules 824 may also cause the processor 810 to receive manual inputs from one or more of user-selectable control options on the time out device, an input device 850 associated with the computing device 804, or the input device 814 deactivate the time out device 202.

The memory 820 may include one or more graphical interface modules 826 that may cause the processor 810 to generate a graphical interface, which may be presented on the display (one of the output devices 816) or that may be provided to a computing device 804 through the network 806. The graphical interface may include text data, image data, user-selectable control options (such as buttons, checkboxes, radio buttons, clickable links, tabs, and so on), or any combination thereof. In some implementations, the graphical interface may be presented within an Internet browser application. In other implementations, the graphical interface may be presented within an application executing on a computing device 804 or on the computing system 802.

The memory 820 may include a time out checklist 828 that may include one or more prompts that define a checklist procedure to be followed. The time out checklist 828 may be configurable to provide a selected workflow or process to be followed. Data corresponding to the time out checklist 828 may be presented within the graphical interface that is sent from the computing system 802 to the computing device 804. In some implementations, the time out checklist 828 may vary depending on the procedure to be performed. Additionally, the items in the time out checklist 828 may be changed by an authorized user, such as to add an item or even to create a new checklist.

The memory 820 may include one or more checklist verification modules 830 that may cause the processor 810 to receive data related to the time out checklist 828. Specifically, a user may interact with the prompts and user-selectable control options presented within the graphical interface to submit data related to the checklist. The checklist verification modules 830 may cause the processor 810 to compare the received data to patient data 840 in the memory 820 to verify that the received data matches data stored in the patient data 840 and optionally other data 842. In some implementations, the other data 842 may include surgical rooms, the names of team members of the medical team, and so on. If the data matches, then the checklist may be completed and verified, and the time out device control module 824 may cause the processor 810 to control the time out device 202 to project an optical indicator indicating that the time out procedure defined by the checklist is complete.

The memory 820 may include one or more sensor modules 832 that may cause the processor 810 to receive data from one or more sensors 818. The data may include optical data, audio data, other data, or any combination thereof. In some implementations, the sensor modules 832 may be configured to capture optical data and may determine a first incision from the optical data, for example, using a trained machine learning algorithm. In response to determining the first incision, the time out device control modules 824 may cause the processor 810 to deactivate the time out device 202. Alternatively, the time out device may be deactivated by the time out device control modules 824 in response to manual input data received from one of the input devices 814 or from a computing device 804.

The memory 820 may include one or more update modules 834 that may cause the processor 810 to receive instructions from the computing device 804 and may update one or more of the modules in the memory 820 based on the instructions. In some implementations, the memory 820 may include one or more focal control modules 836 that may cause the processor 810 to adjust the focus or size of the optical indicator 210 by controlling one or more lenses associated with the time out device 202. The memory 820 may also include one or more other modules 838, which may cause the processor 810 to perform other operations. In an example, the other modules 838 may include alerting instructions that may cause the processor 810 to send a text message, an email message, or another type of electronic alert to an output device 816 (such as a display), a computing device 804, another system, or any combination thereof.

The computing device 804 may include one or more network interfaces 844, which may be configured to send data to and to receive data from the network 806. The computing device 804 may include one or more processors 846 configured to execute instructions and process received data. The computing device 804 may include one or more I/O interfaces 848, which may be coupled to one or more input devices 850 to receive data and to one or more output devices 852 to provide data. The input devices 850 may include a keyboard, a mouse, a stylus, a rollerball, a camera, a microphone, a touch-sensitive interface, another input device, or any combination thereof. The output devices 850 may include a display, a speaker, a printer, another output device, or any combination thereof. In some implementations, an input device 850 and an output device 852 may be combined in the form of a touchscreen 854.

The computing device 804 may include a memory 856 coupled to the processor 846. The memory 856 may store processor-readable instructions and data. In this example, the memory 856 may include one or more operating system modules 858 that may be executed by the processor 846 to enable functionality of various components. The memory 856 may also include a variety of applications 858, such as an Internet browser application or another application that may be configured to display data to an output device 852, to receive data from one or more of the network interface 844 or an input device 850, and to selectively communicate data to the computing system 802.

In some implementations, a user, such as a nurse, a medical technician, an anesthesiologist, a doctor, or another member of the medical team may access a touchscreen 854 to initiate a time out process. The process may be initiated by the user selecting a user-selectable control option presented within a graphical interface on the touchscreen 854. In response to initiation of the time out process, the computing device 804 may send a signal to the computing system 802, which may cause the computing system 802 to generate a graphical interface including data related to the time out checklist 828 and to send the graphical interface to the computing device 804, which may present the graphical interface on the touchscreen 854. Additionally, the computing system 802 may activate the time out device 202 to project an optical indicator 210 onto the surgical site or location on the patient to be treated.

The user may then interact with the time out checklist within the graphical interface via the touchscreen 854, confirming or answering various questions until the checklist is complete, and then submitting the response data. The computing system 802 may receive the response data and verify the response data using the checklist verification modules 830 to determine whether the received data matches data stored in the patient data 840 or other data 842. If the data does not match, the computing system 802 may provide an alert to the computing device 804 to prompt the user to review and correct any discrepancies. Alternatively, if the data matches, the computing system 802 may update the graphical interface presented to the touchscreen 854 and may update the optical indicator by controlling the time out device 202.

In some implementations, once the time out procedure is completed, the sensors 818 may produce signals indicative of optical data. The processor 810 may determine initiation of a surgical procedure by detecting motion near the surgical site or location based on the optical data. In some implementations, the checklist verification modules 830 may cause the processor to determine the start of the procedure from the optical data based on one or more trained models. Alternatively, one or more microphones may capture audio data, which may include a word or command indicative of the start of the procedure. The checklist verification module 830 may cause the processor 810 to determine the start of the surgical procedure based on the word or command or based on the optical data and, in response to determining the start of the surgical procedure, the time out device control modules 824 may cause the processor 810 to deactivate the time out device 202. Other implementations are also possible.

In some implementations, the time out device 202 may be controlled automatically based on the input data and sensor data. In other implementations, the nurse or another team member within the operating room may interact with the touchscreen 854 to control operation of the time out device 202 by interacting with one or more graphical interfaces provided by the computing system 802.

FIG. 9 depicts a flow diagram of a method 900 of automating a time out procedure, in accordance with certain embodiments of the present disclosure. The method 900 may be implemented using the time out device 202 and the computing system 802 of FIGS. 2 and 8.

At 902, the method 900 may include initiating a time out at an input interface of a computing device 804 in a treatment room. In some implementations, the user may interact with one or more user-selectable control options presented within a graphical interface on a touchscreen 854 of the computing device 804 to initiate the surgical time out procedure. Alternatively, the user may interact with a control interface 508 of the time out device 202 or with a remote control 218 associated with the time out device 202.

At 904, the method 900 may include activating a time out device 202 to project a first optical indicator 210(1) onto a surgical site 604 of a patient, where the first optical indicator 210(1) is configured to notify the surgical team to wait. In some implementations, the first optical indicator 210(1) may include the phrase “TIME OUT” in red letters projected onto the surgical site 604. In other implementations, the first optical indicator 210(1) may include a shape, such as a stop sign shape. Other implementations are also possible.

At 906, the method 900 may include determining a checklist in response to initiation of the surgical time out. In some implementations, the computing system 202 may be configured to determine the time out checklist modules 828 to determine a list of details to confirm about the patient, the treatment site, and the procedure prior to starting the procedure. While the method 900 presumes that the procedure is surgical, in some implementations, the procedure may be used in connection with a radiological procedure. Alternatively, the method 900 may be configured for a mechanical procedure or for another type of procedure.

At 908, the method 900 may include providing a series of questions (or prompts) that correspond to the determined checklist to a display (output device 816 or 852 in FIG. 8) in the operating room. The computing system 802 may generate a graphical interface including one or more prompts or queries and including one or more user-selectable control options that may be provided to the output device 816 and accessed by a user via the input interface 814 to answer the prompts. The computing system 802 may send the graphical interface to the computing device 804 for presentation on the touchscreen 854.

At 910, the method 900 may include receiving input data responsive to the series of questions. The user may interact with the graphical interface via the touchscreen 854 to answer the prompts and to submit input data related to the responses to the prompts to the computing device 804. Alternatively, the user may interact with the graphical interface using input device 814 of the computing system 802.

At 912, the method 900 may include comparing the input data to patient data in a database. The computing system 802 may utilize the checklist verification modules 830 to compare the input data to the patient data 840 and optionally to other information.

At 914, if the patient data doesn't match, the method 900 may include providing an alert to the display (touchscreen 854) indicating the mismatched data, at 916. The alert may be presented as a popup message. The method 900 may then return to 906 to determine the checklist in response to the surgical time out.

Returning to 914, if the input data matches the data stored in the patient data 840, the method 900 may include automatically controlling the time out device 202 to project a second optical indicator 210(2) onto the surgical site 604 indicating that the time out is complete and the data is verified, at 918. The second optical indicator 210(2) may include one or more parameters that differ from those of the first optical indicator 210(1). The parameters may include color, shape, size, letters, words, or any combination thereof.

At 920, the method 900 may include determining the treatment is started. The computing system 802 may determine the surgery is started based on input data received from the computing device 804, input data received from a remote control 218, or based on sensor data from the one or more sensors 818.

At 922, the method 900 may include automatically deactivating the time out device 202 in response to determining the start of treatment. The control system 802 may send a control signal to the time out device 202 to turn off the optical indicator 210.

In conjunction with the systems, methods, and devices described above with respect to FIGS. 1-9, a time out device 202 is described that may include an I/O interface 214 configured to receive first input data from one or more of a user selectable button 216 of a control interface 508, a remote control 218, a computing device 220, or a computing system 802. In response to receiving the input data, the time out device 202 may be configured to activate one or more light sources 204 to direct emitted light 208 to produce a first optical indicator 210(1) on a surface. The first optical indicator may include shapes, letters, words, or any combination thereof.

The users may perform a time out procedure to verify various details of the patient, the procedure, the surgical site, and so on. If the data is verified, the time out device 202 may receive second input data from one or more of the user selectable button 216 of the control interface 508, the remote control 218, the computing device 220, or the computing system 802. In response thereto, the time out device 202 may control the one or more light sources to produce a second optical indicator 210(2), which may include one or more parameters that differ from those of the first optical indicator 210(1). In this example, the parameters may include one or more of a color, a size, or the shapes, letters, or words.

Once the surgical procedure is started, the time out device 202 may receive third input data from one or more of the user selectable button 216 of the control interface 508, the remote control 218, the computing device 220, or the computing system 802. In response thereto, the time out device 202 may control the light sources 204 to turn off. Other implementations are also possible.

Although the present invention has been described with reference to preferred embodiments, workers skilled in the art will recognize that changes may be made in form and detail without departing from the scope of the invention.

What is claimed is:

1. A device comprises:
a time out device comprising:
one or more light sources configured to emit light toward a treatment surface to produce an optical indicator;
an interface configured to receive input data to change a status of a time out procedure; and
a microcontroller configured to:
receive first input data from the interface and control the one or more light sources to produce a first optical indicator including one or more first parameters in response to the first input data to indicate a first status;
receive second input data and control the one or more light sources to produce a second optical indicator including one or more second parameters in response to the second input data to indicate a second status; and
receive third input data and turn off the one or more light sources in response to the third input data to indication completion of the time out procedure.

2. The device of claim 1, wherein:
the one or more first parameters include one or more first colors; and
the one or more second parameters include one or more second colors.

3. The device of claim 1, further comprising:
an attachment mechanism configured to releasably couple to one or more of an overhead light, a rail, a handle, or a light assembly; and
an adjustable arm including a proximal end coupled to the attachment mechanism and a distal end coupled to the time out device.

4. The device of claim 1, wherein the time out device is adjustable relative to the treatment surface.

5. The device of claim 1, further comprising:
a base configured to contact a ground surface;
a post including a proximal end and a distal end, the proximal end is coupled to the base and extends from the base at an angle relative to the ground surface; and
an adjustable arm including a proximal end coupled to the distal end of the post and including a distal end coupled to the time out device.

6. The device of claim 1, wherein:
the interface includes one or more buttons accessible by a user; and
the first input data, the second input data, and the third input data correspond to first, second, and third selections, respectively, of one of the one or more buttons by the user.

7. The device of claim 1, wherein:
the interface is coupled to a computing system; and
the interface is configured to receive the input data including the first input data, the second input data, and the third input data from the computing system.

8. The device of claim 1, wherein the time out procedure includes one or more checklist items to be reviewed and verified.

9. A device configured to facilitate a time out verification procedure, the device comprises:
one or more light sources configured to emit light toward a treatment surface to produce an optical indicator; and
a microcontroller configured to control the one or more light sources to provide a first optical indicator at a start of the time out verification procedure and a second optical indicator at successful completion of the time out verification procedure.

10. The device of claim 9, wherein:
the first optical indicator includes one or more of a first color or a first shape;
the second optical indicator includes one or more of a second color or a second shape; and
wherein the first optical indicator and the second optical indicator are programmable.

11. The device of claim 9, further comprising:
an attachment mechanism configured to releasably couple to one or more of an overhead light, a rail, a handle, or a light assembly;
an adjustable arm including a proximal end coupled to the attachment mechanism and a distal end coupled to the time out device; and
wherein the time out device is adjustable relative to the surface.

12. The device of claim 9, further comprising:
a base configured to contact a ground surface;
a post including a proximal end and a distal end, the proximal end is coupled to the base and extends from the base at an angle relative to the ground surface; and
an adjustable arm including a proximal end coupled to the distal end of the post and including a distal end coupled to the time out device.

13. The device of claim 9, further comprising:
an interface coupled to the microcontroller unit, the interface including one or more of a button or a circuit to couple to a computing system to provide input data; and
wherein the microcontroller unit controls the one or more light sources in response to the input data.

14. The device of claim 9, wherein the time out procedure includes one or more checklist items to be reviewed and verified.

15. A device configured to facilitate a time out verification procedure, the device comprises:
one or more light sources configured to emit light toward a treatment surface to produce an optical indicator;
an interface coupled to the one or more light sources to receive input data; and
a microcontroller unit configured to control the one or more light sources to provide a first optical indicator at a start of the time out verification procedure and a second optical indicator at successful completion of the time out verification procedure in response to receiving the input data.

16. The device of claim 15, wherein:
the first optical indicator includes one or more of a first color or a first shape;
the second optical indicator includes one or more of a second color or a second shape; and
wherein the first optical indicator and the second optical indicator are programmable.

17. The device of claim 15, further comprising:
an attachment mechanism configured to releasably couple to one or more of an overhead light, a rail, a handle, or a light assembly;
an adjustable arm including a proximal end coupled to the attachment mechanism and a distal end coupled to the time out device; and
wherein the time out device is adjustable relative to the treatment surface.

18. The device of claim 15, further comprising:
a base configured to rest on a ground surface;
a post including a proximal end and a distal end, the proximal end is coupled to the base and extends from the base at an angle that is substantially perpendicular to the ground surface; and
an adjustable arm including a proximal end coupled to the distal end of the post and including a distal end coupled to the time out device.

19. The device of claim 15, further comprising:
an interface coupled to the microcontroller unit, the interface including one or more of a button or a circuit to couple to a computing system to provide input data; and
wherein the microcontroller unit controls the one or more light sources in response to the input data.

20. The device of claim 15, wherein the microcontroller unit is configured to control the one or more light sources to:
provide the first optical indicator in response to first input data;
provide the second optical indicator in response to second input data; and
deactivate the one or more light sources in response to third input data.
